(19) United States
(12) Patent Application Publication
(10) Pub. No.: US 2022/0133689 A1
(43) Pub. Date: May 5, 2022

(54) TONGKAT ALI EXTRACT PRODUCTION PROCESSES AND USES THEREOF

(71) Applicant: Innovus Pharmaceuticals, Inc., Englewood, CO (US)

(72) Inventor: Bassam Damaj, San Diego, CA (US)

(21) Appl. No.: 17/555,028

(22) Filed: Dec. 17, 2021

Related U.S. Application Data
(63) Continuation of application No. 16/740,194, filed on Jan. 10, 2020, now abandoned.

(51) Int. Cl. A61K 31/366; C07D 493/08; A61K 36/068; A61K 31/404; A61K 36/41; A61K 36/81; A61K 36/67; A61K 36/48

(52) U.S. Cl. CPC A61K 31/366; C07D 493/08; A61K 36/068; A61K 36/48; A61K 36/41; A61K 36/81; A61K 36/67; A61K 31/404

(57) ABSTRACT

Methods for isolating bioactive components from Eurycoma longifolia (Tongkat Ali) are described. In one aspect, the methods comprise concentrating a solution comprising Eurycoma longifolia extract by a continuous-flow evaporation process. Extracts of Eurycoma longifolia and compositions comprising Eurycoma longifolia extract and uses thereof are also provided. The extracts and the compositions are useful for increasing testosterone levels and supporting natural stamina, endurance and strength.

### BACKGROUND

### Field of the Disclosed Technology

The disclosed technology generally relates to a method for isolating bioactive components from Eurycoma longifolia (Tongkat Ali). In one aspect, the methods comprise preparing a Eurycoma longifolia extract by a continuous-flow evaporation process. The disclosed technology further relates to highly purified extracts comprising Eurycoma longifolia, composition comprising extracts of Eurycoma longifolia, and uses of such extracts and compositions. The compositions, in particular, are useful for increasing testosterone levels and supporting natural stamina, endurance, and strength.

### Description of the Related Art

Tongkat Ali, also known as Eurycoma longifolia, pasak bumi, long jack, or Malaysia ginseng, belongs to the Simaroubaceae plants. Alternative names include penawar pahit, bedara pahit, tongkat baginda, petala bumi, setunjang bumi, cay ba binh, and plaa-lai-pueak. Tongkat Ali is a slow-growing tropical rain forest plant, mainly found in Myanmar, Thailand, Malaysia, Vietnam, and China's Hainan Island.

The chemical constituents isolated from different parts of Tongkat Ali are mainly diterpenoids containing iron lignin skeleton and iron indole ketone alkaloids such as eurycomaoside, eurycolactone, eurycomalactone, eurycomanone and pasakbumin-B. In addition, it also contains biphenyl lignin, squalene derivatives, active polysaccharides, glycopeptides and various amino acid.

The roots and/or stems of Tongkat Ali (or extracts of these roots and/or stems) have been used in traditional and folk medicine either as a single herb or as part of multiple herb ingredients to treat dysentery, fever, malaria and sexual problems including male infertility. Tongkat Ali's functions relate to treating hypertension, scabies, jaundice, carbuncles, skin itching, malaria, diarrhea, mouth ulcers, headache and other diseases. Tongkat Ali also has the effect of enhancing male sexual function and treating male sexual dysfunction. For example, European Patent No. EP1952816 discloses a Tongkat Ali extract that has the effect of treating male sexual dysfunction. Malaysian Patent No. MY2006PI03783 disclosed the efficacy of Eurycoma longifolia polar organic solvent extract for promoting growth of sperm, improving sperm quality, and other aspects of the treatment of infertility. Japanese Patent No. JP2012092108 discloses that a Tongkat Ali root extract can be prepared as a medicine for external use for treating male sexual dysfunction. Chinese Patent No. CN102430038 discloses compositions containing Tongkat Ali can increase blood levels of testosterone. Additionally, recent studies have shown that this plant extract contains quassinoids, which also has good anti-tumor and anti-HIV effects.

Tongkat Ali extracts also may be useful for weight control. For example, U.S. Patent Publication No. 2007/0224300 discloses compositions comprising Tongkat Ali extracts that may be used to promote weight loss and help dieters maintain weight loss. Malaysian Patent No. 142166 discloses substances extracted from Eurycoma longifolia useful for treating obesity and diseases associated with obesity.

There are several existing methods to extract eurycomanone from Tongkat Ali. For example, CN103408564B and CN201610743803.5 disclose extraction and purification processes of eurycomanone from Tongkat Ali. However, these techniques extract insufficient biochemically-effective components, take a long time and use a lot of organic solvent. The low production capacity and high production cost make them unsuitable for large-scale production. Accordingly, there is a need for processes that more efficiently prepare Tongkat Ali extract, and that produce an extract that is enriched in eurycomanone.

### SUMMARY

To overcome prior the above-mentioned deficiencies in methods for preparing Tongkat Ali extract and to obtain extracts with higher concentrations of eurycomanone, it is an objective of one aspect of the disclosed technology to develop techniques that are simple, quick, pollution-free, suitable for large-scale production, and entail low energy consumption and low cost.

### BRIEF DESCRIPTION OF THE DRAWINGS

FIG. 1 describes a process of isolating bioactive components from Eurycoma longifolia according to one embodiment.

### DETAILED DESCRIPTION OF THE PREFERRED EMBODIMENTS

In some embodiments, Tongkat Ali extracts comprise quassinoids, which include eurycomanone, 13α,21-dihydroeurycomanone, 13(21)-epoxyeurycomanone, eurycomanol and its glycoside, eurycomaoside and its aglycone, including all their analogues and derivatives; and coumarins, which include 6-methoxycoumarin-7-O-σ-D-glycopyranoside, its other glycosides, analogues and derivatives.

In some embodiments, alternative extraction methods may be used, such as dipping extraction, percolation extraction, reflux extraction, microwave assisted extraction, ultrasonic extraction, supercritical extraction.

The extraction process may be carried out in a variety of solvents. In some embodiments, the solvent may comprise tetrahydrofuran (THF), acetonitrile, a C₁₋₆ alcohol (including but not limited to methanol, ethanol, n-propyl alcohol, isopropyl alcohol, tert-butyl alcohol, n-butyl alcohol, n-pentanol or n-hexanol), toluene, 1,4-dioxane, diethyl ether, methyl tert-butyl ether (MTBE), dimethylformamide (DMF), or dimethylacetamide. In some embodiments, the solvent may comprise water.

In some embodiments, the solvent may comprise a C₁₋₆ alcohol. In some embodiments, the solvent may further comprise water. In some specific embodiments, the solvent may further comprise 1, 2, 3, 4, 5, 6, 7, 8, 9, 10, 15, 20, 25, 30, 35, 40, 45, or 50% v/v water. In some embodiments, the solvent system may further comprise 1% to 10% v/v, 1% to 20% v/v 10% to 30% v/v, 20% to 40% v/v, 15% to 35% v/v, 25% to 35% v/v, 30% to 50% v/v water. In some embodiments, the solvent may be 95/5, 90/10, 85/15, 80/20, 75/25, 70/30, 65/35, 60/40, 55/45, or 50/50% v/v C₁₋₆ alcohol/water. In some embodiments, the C₁₋₆ alcohol may be ethanol. In some embodiments, the solvent may be 70/30% v/v ethanol/water.

The extraction process may be carried out by heating about 750 kg of plant materials with about 6000 L of one of the aforementioned solvents. The extraction process may be carried out at a solvent to plant material ratio of about 8 L/kg. In some embodiments, the extraction process may be carried out at a solvent to plant material ratio of about 0.5, 1, 2, 3, 4, 5, 6, 7, 8, 9, 10, 12.5, 15, 20, 25, 30, 40, or 50 L/kg. In some embodiments, the extraction process may be carried out at a solvent to plant material ratio in the range of 0.1 to 0.5, 0.3 to 1, 0.5 to 2, 1 to 3, 1 to 10, 2 to 4, 3 to 5, 4 to 6, 5 to 7, 6 to 8, 7 to 9, 8 to 10, 9 to 12.5, 5 to 15. 10 to 15, 12.5 to 20, 15 to 25, 20 to 30, 25 to 40, or 30 to 50 L/kg.

In some embodiments, the extraction may be performed at a temperature in the range of about 20° C. to 100° C. In some embodiments, the extraction may be performed at a temperature of about 20, 25, 30, 35, 40, 45, 50, 55, 60, 65, 70, 75, 80, 85, 90, 95, or 100° C. In some embodiments, the extraction may be performed at a temperature in the range of about 60 to 100° C., 70 to 100° C., 80 to 100° C., 90 to 100° C., 60 to 70° C., 60 to 80° C., 60 to 90° C., 65 to 85° C., 65 to 90° C., 70 to 75° C., 70 to 80° C., 75 to 85° C., or 75 to 95° C.

The extraction process may be carried out for an amount of time between about 0.1 to 10 hours. In some embodiments, the extraction process may be carried out for about 0.5, 1.0, 1.5, 2.0, 2.5, 3.0, 3.5, 4.0, 4.5, 5.0, 5.5, 6.0, 6.5, 7.0, 7.5, 8.0, 9.0, or 10.0 hours. In some embodiments, the extraction process may be carried out for an amount of time in the range of 0.1 to 0.5, 0.3 to 1.0, 0.5 to 1.5, 1.0 to 2.0, 1.0 to 3.0, 1.0 to 5.0, 1.5 to 2.5, 2.0 to 3.0, 2.5 to 3.5, 3.0 to 4.0, 3.0 to 5.0, 3.5 to 4.5, 4.0 to 5.0, 4.5 to 5.5, 5.0 to 6.0, 5.5 to 6.5, 6.0 to 7.0, 6.5 to 7.5, 7.0 to 8.0, 7.5 to 9.0, or 8.0 to 10.0 hours, or for an amount of time greater than 10.0 hours. In some embodiments, the extraction process may be carried out for an amount of time between about 1.5 to 2.0 hours.

In some embodiments, the continuous-flow evaporation process is implemented in gas-liquid separators, liquid purifiers, liquid concentrators, desalination systems, or fractional-volatilization separation systems, which enable a stable, continuous, non-bubbling liquid flow, essentially constant surface area/volume ratio, temperature controlled, fractional volatilization of volatile/semi-volatile components in a liquid analyte or component containing sample. The fractional volatilization separator system can be utilized in small scale analytical and large scale chemical purification, concentration and desalinization applications. Continuous rapid removal of residual liquid sample can provide concentrated non-volatile component/analyte solution, and allows quick and easy washout between sequential uses with different liquid samples.

In some embodiments, the continuous-flow evaporation process may be performed at a temperature in the range of about 20° C. to 100° C. In some embodiments, the evaporation process may be performed at a temperature of about 20, 25, 30, 35, 40, 45, 50, 55, 60, 65, 70, 75, 80, 85, 90, 95, or 100° C. In some embodiments, the evaporation process may be performed at a temperature in the range of about 60 to 100° C., 70 to 100° C., 80 to 100° C., 90 to 100° C., 60 to 70° C., 60 to 80° C., 60 to 90° C., 65 to 85° C., 65 to 90° C., 70 to 75° C., 70 to 80° C., 75 to 85° C., or 75 to 95° C.

In some embodiments, the evaporation process may be carried out for about 0.5, 1.0, 1.5, 2.0, 2.5, 3.0, 3.5, 4.0, 4.5, 5.0, 5.5, 6.0, 6.5, 7.0, 7.5, 8.0, 9.0, 10.0, 15.0, 20.0, or 25.0 hours. In some embodiments, the evaporation process may be carried out for an amount of time in the range of 0.1 to 0.5, 0.3 to 1.0, 0.5 to 1.5, 1.0 to 2.0, 1.0 to 5, 1.0 to 10.0, 1.5 to 2.5, 2.0 to 3.0, 2.5 to 3.5, 3.0 to 4.0, 3.0 to 6.0, 3.0 to 10.0, 3.5 to 4.5, 4.0 to 5.0, 4.5 to 5.5, 5.0 to 6.0, 5.5 to 6.5, 6.0 to 7.0, 6.5 to 7.5, 7.0 to 8.0, 7.5 to 9.0, 8.0 to 10.0, 9.0 to 15.0, 10.0 to 20.0, or 15.0 to 25.0 hours, or for an amount of time greater than 25.0 hours. In some specific embodiments, the evaporation process may be carried out for about 6 hours. In some embodiments, alternative concentration methods may be used, such as concentrating at atmospheric pressure, concentrating under reduced pressure and the like, concentrate drying, hot air drying, vacuum dried under reduced pressure, microwave (vacuum) drying, spray drying and other large-scale production and pharmaceutically acceptable drying methods.

In some embodiments, the macro-porous resins may be non-polar macroporous adsorption resin, or low-polarity resin, such as DA-201, D-IO1, LSA-20, HP-10 or AB-8 type macroporous resin. In some embodiments, the macro-porous resins may be non-polar resin, low-polarity resin or middle-polarity resin, the non-polar resin selected from XAD-4, Diaion HP-20, D101, D102, D401, D1, D2, D3, D4, HPD-100 or X-5, the low pole resin selected from D-201 HPD-300 or AB-8, and the middle polarity resin selected from XAD-6, XAD-7 or XAD-8.

In some embodiments, a purification process is performed by combining the desired eluted fraction with a second solvent, and heating at a temperature range of about 70-75° C. for about 8.0 hours to obtain a purified solution.

In some embodiments, the second solvent may comprise tetrahydrofuran (THF), acetonitrile, a C₁₋₆ alcohol (including but not limited to methanol, ethanol, n-propyl alcohol, isopropyl alcohol, tert-butyl alcohol, n-butyl alcohol, n-pentanol or n-hexanol), toluene, 1,4-dioxane, diethyl ether, methyl tert-butyl ether (MTBE), dimethylformamide (DMF), or dimethylacetamide. In some embodiments, the second solvent may comprise water. The second solvent may be the same as, or different than, the first solvent.

In some embodiments, the second solvent may comprise a C₁₋₆ alcohol. In some embodiments, the second solvent may further comprise water. In some specific embodiments, the second solvent may further comprise 1, 2, 3, 4, 5, 6, 7, 8, 9, 10, 15, 20, 25, 30, 35, 40, 45, or 50% v/v water. In some embodiments, the second solvent system may further comprise 1% to 10% v/v, 1% to 20% v/v 10% to 30% v/v, 20% to 40% v/v, 15% to 35% v/v, 25% to 35% v/v, 30% to 50% v/v water. In some embodiments, the second solvent may be 95/5, 90/10, 85/15, 80/20, 75/25, 70/30, 65/35, 60/40, 55/45, or 50/50% v/v C₁₋₆ alcohol/water. In some embodiments, the C₁₋₆ alcohol is ethanol. In some embodiments, the second solvent may be 70/30% v/v ethanol/water.

In some embodiments, the purification process may be performed at a temperature in the range of about 20° C. to 100° C. In some embodiments, the purification process may be performed at a temperature of about 20, 25, 30, 35, 40, 45, 50, 55, 60, 65, 70, 75, 80, 85, 90, 95, or 100° C. In some embodiments, the purification process may be performed at a temperature in the range of about 60 to 100° C., 70 to 100° C., 80 to 100° C., 90 to 100° C., 60 to 70° C., 60 to 80° C., 60 to 90° C., 65 to 85° C., 65 to 90° C., 70 to 75° C., 70 to 80° C., 75 to 85° C., or 75 to 95° C.

In some embodiments, the purification process may be carried out for about 0.5, 1.0, 1.5, 2.0, 2.5, 3.0, 3.5, 4.0, 4.5, 5.0, 5.5, 6.0, 6.5, 7.0, 7.5, 8.0, 9.0, 10.0, 15.0, 20.0, or 25.0 hours. In some embodiments, the purification process may be carried out for an amount of time in the range of 0.1 to 0.5, 0.3 to 1.0, 0.5 to 1.5, 1.0 to 2.0, 1.5 to 2.5, 2.0 to 3.0, 2.5 to 3.5, 3.0 to 4.0, 3.5 to 4.5, 4.0 to 5.0, 4.5 to 5.5, 5.0 to 6.0, 5.5 to 6.5, 6.0 to 7.0, 6.5 to 7.5, 7.0 to 8.0, 7.5 to 9.0, 8.0 to 10.0, 9.0 to 15.0, 10.0 to 20.0, or 15.0 to 25.0 hours, or for an amount of time greater than 25.0 hours.

In some embodiments, a spraying-drying process may be performed by feeding the purified solution into a spraying-drying tank, and collecting a dried powder. The spraying-drying tank may be maintained at a temperature of about 75° C. and having an inlet air temperature of about 165° C. and an outlet air temperature of about 75° C. In some embodiments, the spraying-drying tank may be maintained at a temperature of about 45, 50, 55, 60, 65, 70, 75, 80, 85, 90, or 95° C. In some embodiments, the spraying-drying tank may be maintained at a temperature in the range of 40 to 45, 43 to 50, 45 to 55, 50 to 60, 55 to 65, 60 to 70, 65 to 75, 70 to 80, 75 to 85, 80 to 90, or 85 to 95° C., or at a temperature greater than 95° C. In some embodiments, the spraying-drying tank may have an inlet air temperature of about 135, 140, 145, 150, 155, 160, 165, 170, 175, 180, 185, 190, 195, or 200° C. In some embodiments, the spraying-drying tank may have an inlet air temperature in the range of 135 to 143, 140 to 145, 143 to 150, 145 to 155, 150 to 160, 155 to 165, 160 to 170, 165 to 175, 710 to 180, 175 to 185, 180 to 190, 185 to 195, or 190 to 200° C., or have an inlet air temperature greater than 200° C. In some embodiments, the spraying-drying tank may have an outlet air temperature of about 45, 50, 55, 60, 65, 70, 75, 80, 85, 90, or 95° C. In some embodiments, the spraying-drying tank may have an outlet air temperature in the range of 40 to 45, 43 to 50, 45 to 55, 50 to 60, 55 to 65, 60 to 70, 65 to 75, 70 to 80, 75 to 85, 80 to 90, or 85 to 95° C., or have an outlet air temperature greater than 95° C.

In some embodiments, the presence and contents of quassinoids and coumarins, including their analogues and derivatives, as such analogues and derivatives may be present in the Tongkat Ali plant material or may be a by-product generated by the production method. These components may be present in extracts, dried powder, compositions and/or pharmaceutical products, are analyzed by chromatographic processes including reversed phase high-performance liquid chromatography (HPLC) and mass spectroscopy (MS) and identified by ultraviolet, infrared, mass spectroscopies and nuclear magnetic resonance and X-ray diffraction analysis.

In some embodiments, the compositions and/or pharmaceutical products comprise a high percentage of the compound eurycomanone. In some embodiments, the compositions and/or pharmaceutical products comprise a high percentage of bioactive components of Eurycoma longifolia.

In some embodiments, a composition is provided comprising Eurycoma longifolia extract. In some embodiments, the composition is formulated for oral administration. For example, the composition may be prepared as a capsule, tablet, powder, or sachet. In some embodiments, a unit dose of the composition may be one, two, three, four, or more capsules in some specific embodiments, a unit dose of the composition may be two capsules.

In some embodiments, the composition comprises Eurycoma longifolia extract. In some embodiments, the composition is in the form of a capsule for oral administration. In some embodiments, the amount of Eurycoma longifolia extract in the composition may be about 50, 60, 70, 80, 90, 100, 125, 150, 175, 200, 225, 250, 275, 300, 350, 400, 450, or 500 mg or within a range defined by any two of the aforementioned values per capsule. Approximately 10 wt. %, 15 wt. %, 20 wt. %, 25 wt. %, 30 wt. %, 35 wt. %, or 40 wt. % Eurycoma longifolia extract present in the composition are contemplated within the scope of the invention.

In some embodiments, the composition further comprises Fenugreek seed extract. In some embodiments, the amount of Fenugreek seed extract in the composition may be about 50, 60, 70, 80, 90, 100, 125, 150, 175, 200, 225, 250, 275, 300, 350, 400, 450, or 500 mg or within a range defined by any two of the aforementioned values per capsule. Approximately 5 wt. %, 10 wt. %, 15 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, or 35 wt. % Fenugreek seed extract present in the composition are contemplated within the scope of the invention.

In some embodiments, the composition further comprises Cordyceps. Cordyceps is a fungus that is known to live on certain caterpillars in the high mountain regions of China. Cordyceps may improve immunity, may have anticancer activity, may improve athletic performance, and may treat male sexual problems. In some embodiments, the amount of Cordyceps in the composition may be about 10, 20, 30, 40, 50, 60, 70, 80, 90, 100, 125, 150, 175, 200, 225, 250, 275, 300, 350, or 400 or within a range defined by any two of the aforementioned values per capsule. Approximately 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % Cordyceps present in the composition are contemplated within the scope of the invention.

In some embodiments, the composition further comprises Rhodiola rosea extract. In some specific embodiments, the composition further comprises Rhodiola rosea root extract. In some embodiments, the amount of Rhodiola Rosea extract in the composition may be about 10, 20, 30, 40, 50, 60, 70, 80, 90, 100, 125, 150, 175, 200, 225, 250, 275, 300, 350, or 400 or within a range defined by any two of the aforementioned values per capsule. Approximately 1 wt %. 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % Rhodiola rosea extract present in the composition are contemplated within the scope of the invention.

In some embodiments, the composition further comprises Ashwagandha extract. In some specific embodiments, the composition further comprises Ashwagandha root extract. In some embodiments, the amount of Ashwagandha extract in the composition may be about 10, 20, 30, 40, 50, 60, 70, 80, 90, 100, 125, 150, 175, 200, 225, 250, 275, 300, 350, or 400 or within a range defined by any two of the aforementioned values per capsule. Approximately 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % Ashwagandha extract present in the composition are contemplated within the scope of the invention.

In some embodiments, the composition further comprises diindolylmethane. In some embodiments, the amount of diindolylmethane in the composition may be about 10, 20, 30, 40, 50, 60, 70, 80, 90, 100, 125, or 150, or within a range defined by any two of the aforementioned values per capsule. Approximately 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or 15 wt. % diindolylmethane present in the composition are contemplated within the scope of the invention.

In some embodiments, the composition further comprises black pepper extract. In some embodiments, the amount of black pepper extract in the composition may be about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, or 20 mg or within a range defined by any two of the aforementioned values per capsule. Approximately 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 1.6 wt. %, 1.7 wt. %, 1.8, wt. % 1.9 wt. %, or 2.0 wt. % black pepper extract present in the composition are contemplated within the scope of the invention.

In some embodiments the composition may comprise a mass ratio of about 4:1, 4:2, 4:3, 3:1, 2:1, 1:1, 1:2, 1:3, or 1:4 of Eurocma longifolia extract to fenugreek extract. In some embodiments the composition may comprise Rhodiola rosea root extract and ashwanganda root extract in a mass ratio of about 5:1, 4:1, 3:1:2:1, 1:1, 1:2, 1:3, 1:4 or 1:5.

In certain embodiments, the composition may comprise vitamin B6, magnesium oxide, zinc oxide, gelatin, or water; or a combination thereof.

In some embodiments, the pharmaceutical products comprise about 400 mg of Eurycoma longifolia extract in 2 capsules. In some embodiments, the pharmaceutical products comprise about 400 mg of Eurycoma longifolia extract, about 300 mg of fenugreek seed extract, about 100 mg of Cordyceps mycelium, about 100 mg of Rhodiola rosea extract, about 100 mg of Ashwagandha extract, about 66 mg of diindolylmethane (DIM), about 14 mg of black pepper extract, about 20 mg of vitamin B6, about 100 mg of magnesium, and about 30 mg of zinc in 2 capsules. In some embodiments, the pharmaceutical products comprise about 200 mg of Eurycoma longifolia extract per capsule. In some embodiments, the pharmaceutical products comprise about 200 mg of Eurycoma longifolia extract, about 150 mg of fenugreek seed extract, about 50 mg of Cordyceps mycelium, about 50 mg of Rhodiola rosea extract, about 50 mg of ashwagandha extract, about 33 mg of diindolylmethane (DIM), and about 7 mg of black pepper extract per capsule. In some embodiments, each capsule may further comprise about 10 mg of vitamin B6, about 50 mg of magnesium, and about 15 mg of zinc.

In some embodiments, the vitamin B6 is in the form of pyridoxine hydrochloride. In some embodiments, the magnesium is in the form of magnesium oxide. In some embodiments, the zinc is in the form of zinc oxide. In some embodiments, black pepper extract is in the form of BioPerine®. In some embodiments, the Eurycoma longifolia extract is obtained from the root. In some embodiments, the Rhodiola rosea extract is obtained from the root. In some embodiments, the ashwagandha extract is obtained from the root.

In some embodiments, the pharmaceutical products comprise 400 mg of Eurycoma longifolia extract per 1230 mg of pharmaceutical product. In some embodiments, the pharmaceutical products comprise about 32.5% of Eurycoma longifolia extract. In some embodiments, the pharmaceutical products may comprise about 2.5%, 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 92.5%, 95%, 97.5%, or 100% of Eurycoma longifolia extract. In some embodiments, the pharmaceutical products may comprise about 0.1 to 5%, 3 to 10%, 5 to 15%, 10 to 20%, 15 to 25%, 20 to 30%, 25 to 30%, 27.5 to 33%, 30 to 35%, 33 to 37%, 35 to 40%, 37 to 43%, 40 to 45%, 43 to 50%, 45 to 55%, 50 to 60%, 55 to 65%, 60 to 67%, 65 to 70%, 67 to 75%, 70 to 80%, 75 to 85%, 80 to 90%, 85 to 95%, or 90 to 100% of Eurycoma longifolia extract.

In some embodiments, the Eurycoma longifolia extract may comprise more than about 1%, 2%, 3%, 5%, 7%, 10%, 15%, 20%, 25%, 30%, 40%, 50%, 60%, 70%, 80%, or 90% of the compound eurycomanone by weight. In some embodiments, the Eurycoma longifolia extract may comprise at least about 1%, 2%, 3%, 5%, 7%, 10%, 15%, 20%, 25%, 30%, 40%, 50%, 60%, 70%, 80%, or 90% of the compound eurycomanone by weight. In some embodiments, the Eurycoma longifolia extract prepared according to the disclosed methods may comprise about 1 to 3%, 2 to 5%, 3 to 10%, 5 to 15%, 10 to 20%, 15 to 25%, 20 to 30%, 25 to 30%, 27.5 to 33%, 30 to 35%, 33 to 37%, 35 to 40%, 37 to 43%, 40 to 45%, 43 to 50%, 45 to 55%, 50 to 60%, 55 to 65%, 60 to 67%, 65 to 70%, 67 to 75%, 70 to 80%, 75 to 85%, 80 to 90%, 85 to 95%, or 90 to 100% of the compound eurycomanone by weight. Such extracts may comprise percentages of eurycomanone that are greater than could be obtained by prior processes

In some embodiments, the dried powder prepared according to the disclosed methods (after spray-drying) comprise more than about 5% of the compound eurycomanone by weight. In some embodiments, the dried powder prepared according to the disclosed methods may comprise more than about 1%, 2%, 3%, 5%, 7%, 10%, 15%, 20%, 25%, 30%, 40%, 50%, 60%, 70%, 80%, or 90% of the compound eurycomanone by weight. In some embodiments, the dried powder prepared according to the disclosed methods may comprise about 1 to 3%, 2 to 5%, 3 to 10%, 5 to 15%, 10 to 20%, 15 to 25%, 20 to 30%, 25 to 30%, 27.5 to 33%, 30 to 35%, 33 to 37%, 35 to 40%, 37 to 43%, 40 to 45%, 43 to 50%, 45 to 55%, 50 to 60%, 55 to 65%, 60 to 67%, 65 to 70%, 67 to 75%, 70 to 80%, 75 to 85%, 80 to 90%, 85 to 95%, or 90 to 100% of the compound eurycomanone by weight.

In some embodiments, the pharmaceutical products comprise more than about 1.62% of the compound eurycomanone by weight. In some embodiments, the pharmaceutical products may comprise more than about 0.25, 0.50, 0.75, 1.00, 1.25, 1.50, 1.62, 1.75, 2.00, 2.25, 2.50, 2.75, 3.00, 3.25, 3.50, 3.75, 4.00, 4.25, 4.50, 4.75, 5.00, 5.50, 6.00, 6.50, 7.00, 10.00, 15.00, 20.00, 25.00, 30.00, 40.00, 50.00, 60.00, 70.00, 80.00, or 90.00% of the compound eurycomanone by weight. In some embodiments, the pharmaceutical products may comprise about 0.1 to 0.5%, 0.25 to 0.75%, 0.5 to 1.00%, 0.75 to 1.25%, 1.00 to 1.50%, 1.25 to 1.62%, 1.50 to 1.75%, 1.62 to 2.00%, 1.75 to 2.25%, 2.00 to 2.50%, 2.25 to 2.75%, 2.50 to 3.00%, 2.75 to 3.25%, 3.00 to 3.50%, 3.25 to 3.75%, 3.50 to 4.00%, 3.75 to 4.25%, 4.00 to 4.50%, 4.25 to 4.75%, 4.50 to 5.00%, 4.75 to 5.50%, 5.00 to 6.00%, 5.50 to 6.50%, 6.00 to 7.00%, 6.50 to 10.00%, 7.00 to 15.00%, 10.00 to 20.00%, 15.00 to 25.00%, 20.00 to 30.00%, 25.00 to 40.00%, 30.00 to 50.00%, 40.00 to 60.00%, 50.00 to 70.00%, 60.00 to 80.00%, 70.00 to 90.00%, or 90.00 to 100.00% of the compound eurycomanone by weight.

In some embodiments, the compositions and/or pharmaceutical products may comprise clinically acceptable powders, granules, capsules, pills, tablets or other forms. In some embodiments, the dried powder may be added to clinically acceptable carriers, such as tablets, pills, or granules. In some embodiments, Tongkat Ali extracts made be administered via pills, granules, oral liquid, capsules, tablets, syrups, injections, or other clinically acceptable forms.

In some embodiments, pharmaceutically acceptable carriers may include, but are not limited to: saccharose, magnesium oxide, zinc oxide, dextrin, starch, lactose, mannitol, xylitol, chitosan, chitosan, Bifidobacterium sugar, talc, sodium carboxymethylcellulose (CMS-Na), microcrystalline cellulose (MCC), silica powder, α-cyclodextrin, β-cyclodextrin, polyvinylpyrrolidone (povidone), hydroxypropyl cellulose, polyethylene glycol (PEG).

In some embodiments, dyes may be added to the solutions and/or compositions, such as iron oxide yellow and/or red iron oxide and/or titanium dioxide, for the purpose of color matching, and may be used alone or in combination with the pharmaceutically acceptable carrier.

In some embodiments, the solutions and/or compositions provided herein may comprise a pharmaceutical carrier, diluent, co-solvent, emulsifier, penetration enhancer, preservative, emollient, or a combination thereof. Acceptable carriers or diluents for therapeutic use are well-known in the pharmaceutical art, and are described, for example, in Remington's Pharmaceutical Sciences, 18th Ed., Mack Publishing Co., Easton, Pa. (1990), which is incorporated herein by reference in its entirety.

Preservatives, stabilizers, dyes, fragrances, and the like may be provided in the solutions and/or compositions. For example, sodium benzoate, ascorbic acid, benzyl benzoate, and esters of p-hydroxybenzoic acid may be added as preservatives. In addition, antioxidants and suspending agents may be used. In one or more of the contemplated embodiments, alcohols, esters, sulfated aliphatic alcohols, and the like may be used as surface active agents; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

In certain embodiments, the solutions and/or compositions may comprise sorbitol, isopropyl alcohol, propylene glycol, butylated hydroxytoluene, triethanolamine, benzyl alcohol, benzyl benzolate, PEG 40-hydrogenated castor oil, acrylate/C₁₀₋₃₀ alkyl acrylate crosspolymer, disodium EDTA, or water; or a combination thereof.

The terms “pharmaceutically acceptable carrier” or “pharmaceutically acceptable excipient,” as used herein, include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “excipient,” as used herein, refers to an inert or relatively inert substance that is added to a pharmaceutical composition to impart certain properties to the composition including, without limitation, improved or desired bulk, consistency, stability, binding ability, lubrication, disintegrating ability, etc. A “diluent” is a type of excipient.

### EXAMPLES

Materials used in isolating bioactive components from Eurycoma longifolia described herein may be made by known methods or are commercially available. It is also possible to make use of variants that are known to those of ordinary skill in this art, but are not mentioned here in greater detail. The skilled artisan, given the literature and this disclosure, is well equipped to prepare the formulations of the instant application.

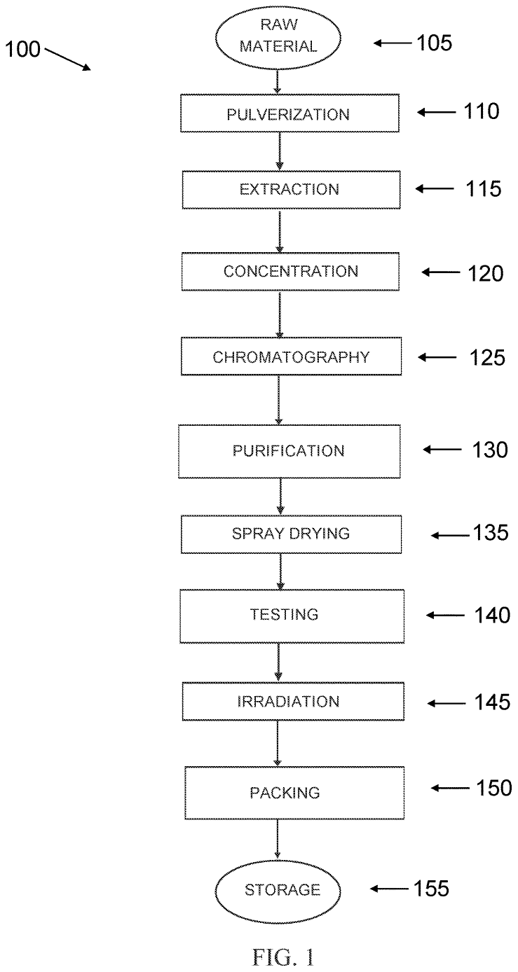

FIG. 1 describes a process 100 for isolating bioactive components from Eurycoma longifolia.

### Example 1: Processing of Raw Materials

Referring to FIG. 1, Tongkat Ali raw materials 105 are purchased from the local importer where they are clean and cut into short blocks. These blocks are spliced and thereafter pulverized in step 110 into coarse chips and weight for use in the production. No further washing and drying is needed. Materials received from the importer are subjected to sampling test using HPLC to verify eurycomanone presence.

In an embodiment for producing a 150 kg Tongkat Ali extract order, 14,250 kg of pulverized coarse chops of Tongkat Ali roots are prepared.

### Example 2A: Extraction of Processed Materials

Referring to FIG. 1, in step 115 the pulverized coarse chips of Tongkat Ali is loaded into the extraction tank and added with solvent (70% ethanol/30% water) and boiled to 70-75° C. temperature and allowed to circulate for 1.5-2.0 hours before the supernatant is drained.

In an embodiment for producing a 150 kg Tongkat Ali extract order, the process cycle data is shown in Tables 1-4 below. A total of three (3) extraction tanks were used to extract the 14,250 kg in three (3) production batches. Each production batch is a “Run” with simultaneous production using three (3) tanks. Each Run consists of three “Cycles” in reusing the Tongkat Ali materials.

TABLE 1
Conditions for Production Batch Runs

| Description | In | Out | Temp | Duration |
|---|---|---|---|---|
| 1) Fill TA materials | 0600 hr | 0630 hr | Ambient | 30 Min |
| 2) Add Solvent | 0630 hr | 0700 hr | Ambient | 30 Min |
| 3) Boiling | 0700 hr | 0800 hr | Reach 80° C. | 60 Min |
| 4) Circulation | 0800 hr | 1000 hr | 70-75° C. | 120 Min |
| 5) Collect Supernatant | 1000 hr | 1100 hr | 70-75° C. | 60 Min |
| End 1^{st} Cycle | | | | 300 Min/5 Hrs |
| 6) Add Solvent | 1100 hr | 1130 hr | Ambient | 30 Min |
| 7) Boiling | 1130 hr | 1200 hr | Reach 80° C. | 30 Min |
| 8) Circulation | 1200 hr | 1330 hr | 70-75° C. | 90 Min |
| 9) Collect Supernatant | 1330 hr | 1430 hr | 70-75° C. | 60 Min |
| End 2^{nd} Cycle | | | | 210 Min/3.5 Hrs |
| 10) Add Solvent | 1430 hr | 1500 hr | Ambient | 30 Min |
| 11) Boiling | 1500 hr | 1530 hr | Reach 80° C. | 30 Min |
| 12) Circulation | 1530 hr | 1700 hr | 70-75° C. | 90 Min |
| 13) Collect Supernatant | 1700 hr | 1800 hr | 70-75° C. | 60 Min |
| End of 3^{rd} Cycle | | | | 210 Min/3.5 Hrs |
| TOTAL RUN PROCESS | | | | 720 Min/12 Hrs |
| [TA Materials REUSED | | | | |
| for 2^{nd} & 3^{rd} Cycles] | | | | |

TABLE 2
Conditions for Production Batches

| | TA | | | Solvent | Supernatant |
|---|---|---|---|---|---|
| CYCLE | LOAD | Total Time | | (70%/30%) IN | OUT |
| Tank #1 | | | | | |
| 1st | 750 kg | 5 | Hrs | 6,000 L | 5,500 L |
| 2nd | Reused | 3.5 | Hrs | 3,000 L | 3,000 L |
| 3rd | Reused | 3.5 | Hrs | 3,000 L | 3,000 L |
| Total | 750 kg | 12 | Hrs | 12,000 L | 11,500 L |
| Tank #2 | | | | | |
| 1st | 750 kg | 5 | Hrs | 6,000 L | 5,500 L |
| 2nd | Reused | 3.5 | Hrs | 3,000 L | 3,000 L |
| 3rd | Reused | 3.5 | Hrs | 3,000 L | 3,000 L |
| Total | 750 kg | 12 | Hrs | 12,000 L | 11,500 L |
| Tank #3 | | | | | |
| 1st | 750 kg | 5 | Hrs | 6,000 L | 5,500 L |
| 2nd | Reused | 3.5 | Hrs | 3,000 L | 3,000 L |
| 3rd | Reused | 3.5 | Hrs | 3,000 L | 3,000 L |
| Total | 750 kg | 12 | Hrs | 12,000 L | 11,500 L |
| TOTAL | 2,250 kg | 12 | Hrs | 36,000 L | 34,500 L |
| BATCH | | | | | |

TABLE 3
Weight and Volume Yields for Production Days

| | | | | | | | Total |
|---|---|---|---|---|---|---|---|
| Day | Hours | Batch | TA Used | Tank #1 | Tank #2 | Tank #3 | Supernatant |
| 10/10 | 12 Hr | 1A | 2,250 kg | 11,500 L | 11,500 L | 11,500 L | 34,500 L |
| 11/10 | 12 Hr | 1B | 2,250 kg | 11,500 L | 11,500 L | 11,500 L | 34,500 L |
| 12/10 | 12 Hr | 1C | 2,250 kg | 11,500 L | 11,500 L | 11,500 L | 34,500 L |
| Total 1 | | | 6,750 kg | 34,500 L | 34,500 L | 34,500 L | 103,500 L |
| 13/10 | 12 Hr | 2A | 2,250 kg | 11,500 L | 11,500 L | 11,500 L | 34,500 L |
| 14/10 | 12 Hr | 2B | 2,250 kg | 11,500 L | 11,500 L | 11,500 L | 34,500 L |
| 15/10 | 12 Hr | 2C | 2,250 kg | 11,500 L | 11,500 L | 11,500 L | 34,500 L |
| Total | | | 6,750 kg | 34,500 L | 34,500 L | 34,500 L | 103,500 L |
| 18/10 | 12 Hr | 3A | 750 kg | 11,500 L | Not used | Not used | 11,500 L |
| Total | | | 750 kg | 11,500 L | — | — | 11,500 L |
| TOTAL | | | 14,250 kg | 80,500 L | 69,000 L | 69,000 L | 218,500 L |

TABLE 4
Summary Data - Extraction

| | | SOLVENT | | |
|---|---|---|---|---|
| TA | NO. OF RUNS | USED | SUPERNATANT | YIELD |
| USED | @ 750 kg/RUN | 16:1 RATIO | YIELD | % |
| 14,250 kg | 19 | 228,000 L | 218,500 L | 95% |

### Example 2B: Alternative Method for Extraction of Processed Materials

Alternatively, referring to FIG. 1, in step 115 the pulverized coarse chips of Tongkat Ali is loaded into the extraction tank and added with solvent (90% ethanol/10% water) and boiled to 80-90° C. temperature and allowed to circulate for 3.5-5.0 hours before the supernatant is drained. In an embodiment for producing a 150 kg Tongkat Ali extract order, a total of three (3) extraction tanks were used to extract the 14,250 kg in three (3) production batches.

### Example 3A: Evaporation/Concentration of Supernatants

Referring to FIG. 1, in step 120 the supernatant collected from the extraction process is feed into the Evaporation/Concentration tank on a continuous flow and circulated for six (6) hours at 70-75° C. temperature and the total solids (thickened supernatant) is collected.

In an embodiment for producing a 150 kg Tongkat Ali extract order, the process cycle data is shown in Table 5 below. Three (3) Evaporation-Concentration tanks were used to feed the supernatant collected from the Extraction process. These were on a continuous flow as the tanks capacity are available from the draining of the thickened supernatant.

TABLE 5

| Flow | Evap #1 | | Evap #2 | | Evap #3 | | Total | |
|---|---|---|---|---|---|---|---|---|
| Supernatant In | 3,834 | L | 3,833 | L | 3,833 | L | 11,500 | L |
| Thickened | 234 | kg | 233 | kg | 233 | kg | 700 | kg |
| Supernatant Out | | | | | | | | |
| Cycle Time | 6 | hr | 6 | hr | 6 | hr | | |
| Temperature | 70-75° | C. | 70-75° | C. | 70-75° | C. | | |
| 19 Cycles IN | 72,846 | L | 72,827 | L | 72,827 | L | 218,500 | L |
| 19 Cycles OUT | 4,446 | kg | 4,427 | kg | 4,427 | kg | 13,300 | kg |

| | | | |
|---|---|---|---|
| Total Cycle Time | 108 Hr/2 Day | 108 Hr/2 Day | 108 Hr/2 Day |

### Example 3B: Alternative Methods for Evaporation/Concentration of Supernatant

Alternatively, referring to FIG. 1, in step 120 the supernatant collected from the extraction process is feed into the Evaporation/Concentration tank on a continuous flow and circulated for ten (10) hours at 70-85° C. temperature and the total solids (thickened supernatant) is collected. In an embodiment for producing a 150 kg Tongkat Ali extract order, three (3) Evaporation-Concentration tanks were used to feed the supernatant collected from the Extraction process. These were on a continuous flow as the tanks capacity are available from the draining of the thickened supernatant.

### Example 4: Column Chromatography/Separation

Referring to FIG. 1, in step 125 the thickened supernatant is feed into the column that is prefilled with non-toxic macro-porous resins/molecular sieve, and distilled water is added. Elution of impurities are at intervals during a 6 hours at ambient temperature, and the separated supernatant is collected.

In an embodiment for producing a 150 kg Tongkat Ali extract order, the process cycle data is shown in Table 6 below. Two (2) Columns were used to feed through 13,300 kg of thickened Supernatant collected from the Evaporation-Concentration process. The feed through rate is approximately 165 kg/hour and process takes two (2) days. Each column is filled with:

i. 200 kg of Macro-porous Resin/Molecular Sieve (Non Toxic)

ii. These resins are cleaned after each 1,000 kg of thickened Supernatant is feed through.

iii. The thickened Supernatant is feed in.

iv. Distilled water totaling 4,000 L is added in flushing-separation process for each 1,000 kg of thickened Supernatant.

TABLE 6
Process Cycle Data

| Flow | Column #1 | Column #2 | Total |
|---|---|---|---|
| Feed | 6,650 kg | 6,650 kg | 13,300 kg |
| Add distilled water (4,000 L | 26,600 L | 26,600 L | 53,200 L |
| H₂O/1,000 kg Supernatant | | | |
| Collection | 15,000 L | 15,000 L | 30,000 L |

### Example 5A: Recovery/Purification

Referring to FIG. 1, in step 130 the separated supernatant is feed into the recovery tank where solvent (70% ethanol/30% water) is added to circulate for 8 hours at 70-75° C. temperature, and the purified supernatant is collected thereafter.

In an embodiment for producing a 150 kg Tongkat Ali extract order, the process cycle data is shown in Table 7 below. The separated supernatant after elution in the Column Chromatography process is then feed into the Recovery-Purification process tank. Two (2) tanks are used. Flow process rate is 600 kg per hour and process take 2 days.

TABLE 7

| | Recovery | | Recovery | | | |
|---|---|---|---|---|---|---|
| Flow | #1 | | #2 | | Total | |
| Feed | 15,000 | L | 15,000 | L | 30,000 | L |
| Temperature | 70-75° | C. | 70-75° | C. | 70-75° | C. |
| Capacity 1,000 L tank | 25 | hours | 25 | hours | 25 | hours |
| with continuous feed | | | | | | |
| at 600 L flow rate | | | | | | |
| Thickened Supernatant | 600 | kg | 600 | kg | 1,200 | kg |

### Example 5B: Alternative Method for Recovery/Purification

Alternatively, referring to FIG. 1, in step 130 the separated supernatant is feed into the recovery tank where solvent (60% ethanol/40% water) is added to circulate for 20 hours at 60-65° C. temperature, and the purified supernatant is collected thereafter. In an embodiment for producing a 150 kg Tongkat Ali extract order, the separated supernatant after elution in the Column Chromatography process is then feed into the Recovery-Purification process tank. Two (2) tanks are used. Flow process rate is 600 kg per hour and process take 2 days.

### Example 6: Spray Drying

Referring to FIG. 1, in step 135 the purified supernatant is feed into the spray drying tank with an inlet air temperature of 165° C. and an outlet temperature of 75° C., while the internal tank is maintain at 75° C. The flow rate through the funnel and nozzle outlet is averaging 3 kg powder per hour. The process cycle data is shown in Table 5 below.

In an embodiment for producing a 150 kg Tongkat Ali extract order, the process cycle data is shown in Table 8, below. The thickened supernatant collected from the recovery process are then feed into the spray drying machine from each production batch.

TABLE 8

| Flow rate on yield | 3 kg per hour/52.6 hours |
|---|---|
| Feed | 1,200 kg |
| Dried Powder Yield | 158 kg |
| Ratio | 7.59:1 (13%) |

### Example 7: Summary Analysis

In an embodiment for producing a 150 kg Tongkat Ali extract order, the summary data is shown in Table 9, below.

TABLE 9

| Flow | Start | | Add | End | | Ratio/% |
|---|---|---|---|---|---|---|
| Extraction | 14,250 | kg TA | 228,000 L | 218,500 | L | 16:1 |
| | | | Solvent | | | |
| Evaporation/Concentration | 218,500 | L | No Additives | 13,300 | kg | 6% |
| Supernatant | | | | | | |
| Column Chromatography | 13,300 | kg | 53,200 L | 30,000 | L | 45% |
| | | | Distilled | | | |
| | | | Water | | | |
| Recovery/Purification | 30,000 | L | No Additives | 1,200 | kg | 4% |
| Spray Drying - Extracted | 1,200 | kg | No additives | 158 | kg | 13.2% |
| Powder | | | | | | |
| Net Ratio (Use of TA) | 14,250 | kg | | 158 | kg | 90:1 |

Referring to FIG. 1, in step 140 the dried powder Tongkat Ali extract is tested using HPLC for its eurycomanone content. In an embodiment, the dried powder is found to comprise more than 5% eurycomanone level.

### Example 8: Irradiation

Referring to FIG. 1, in step 145 the dried powder Tongkat Ali extract is vacuum packed at a Controlled Facility into 1 kg bags, and loaded into 25 kg containers, sealed and sent to a controlled facility (e.g., Hunan Irradiation Center) for irradiation process using Cobalt-60 method.

### Example 9: Representative Tongkat Ali Extract Compositions

Representative formulations according to the invention are shown in Table 1 below, with the amounts for “broad,” “intermediate,” and “preferred” ranges.

TABLE 10
Representative Tongkat Ali Compositions (per capsule)

| | Broader | Narrower | Preferred |
|---|---|---|---|
| | Range | Range | Range |
| Component | mg | mg | mg |
| Tongkat Ali Extract | 50-500 | 100-300 | 200 |
| Fenugreek Seed Extract | 50-250 | 100-200 | 150 |
| Cordyceps | 5-100 | 25-75 | 50 |
| Rhodiola Rosea Root Extract | 5-100 | 25-75 | 50 |
| Ashwagandha Root Extract | 5-100 | 25-75 | 50 |
| diindolylmethane | 10-100 | 20-40 | 33 |
| Black pepper extract | 1-20 | 2-10 | 7 |

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosed technology.

1-23. (canceled)

24. A composition comprising 10% eurycomanone by weight.

25. The composition of claim 24, which is formulated for oral administration.

26. The composition of claim 25, wherein the composition is a component of a capsule, tablet, powder, or sachet dosage form.

27. A method of making a eurycomanone composition, comprising:
preparing an extract of Eurycoma longifolia comprising eurycomanone in a first solvent;
concentrating the extract;
separating impurities from the eurycomanone in the extract;
purifying the extract in a second solvent; and
drying the extract to produce a powder;
wherein the powder contains at least 5% eurycomanone by weight.

28. The method of claim 27, wherein the first solvent comprises about 70% ethanol by volume.

29. The method of claim 27, wherein the Eurycoma longifolia is extracted at about 70° C. to about 75° C. for about 1.5 to about 2 hours.

30. The method of claim 27, wherein the first solvent comprises about 90% ethanol by volume.

31. The method of claim 30, wherein the Eurycoma longifolia is extracted at about 80° C. to about 90° C. for about 3.5 to about 5 hours.

32. The method of claim 27, wherein the Eurycoma longifolia is extracted three times.

33. The method of claim 27, wherein the extract is concentrated by continuous flow evaporation.

34. The method of claim 33, wherein the extract is concentrated at about 70° C. to about 75° C. for about 6 hours.

35. The method of claim 33, wherein the extract is concentrated at about 70° C. to about 85° C. for about 10 hours.

36. The method of claim 27, wherein the impurities are separated from the eurycomanone by column chromatography.

37. The method of claim 27, wherein the second solvent comprises about 70% ethanol by weight.

38. The method of claim 37, wherein the extract is purified at about 70° C. to about 75° C. for about 8 hours.

39. The method of claim 27, wherein the second solvent comprises about 60% ethanol by weight.

40. The method of claim 39, wherein the extract is purified at about 60° C. to about 65° C. for about 20 hours.

41. The method of claim 27, wherein the extract is dried by spray drying.

42. The method of claim 27, wherein the powder comprises at least 10% eurycomanone by weight.

43. The method of claim 27, further comprising, after drying, formulating the composition in a capsule, tablet, powder, or sachet for oral administration.

44. A method of making a eurycomanone composition, comprising:
I. extracting Eurycoma longifolia by heating in a solution of 70% ethanol by volume at 70° C. to 80° C. for about 1.5 to 2 hours;
II. concentrating the resulting extract by continuous flow evaporation at 70° C. to 75° C. for about 6 hours;
III. separating impurities from the concentrated extract by column chromatography;
IV. purifying the extract by heating in a solution of 70% ethanol by weight at 70° C. to 75° C. for about 8 hours; and
V. spray drying the purified extract to produce a powder; wherein:
the Eurycoma longifolia comprises eurycomanone; and
the powder contains at least 10% eurycomanone by weight.

45. The method of claim 44, wherein step I is repeated three times prior to concentrating in step II.

46. The method of claim 45 further comprising, after step V, formulating the composition in a capsule, tablet, powder, or sachet for oral administration.
